(19) United States
(12) Patent Application Publication
(10) Pub. No.: US 2007/0025941 A1
(43) Pub. Date: Feb. 1, 2007

(54) Liquid lip gloss compositions with enhanced shine

(75) Inventors: Valerie Robert, Scotch Plains, NJ (US); Padraig McDermott, Westfield, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'ORÉAL, Paris (FR)

(21) Appl. No.: 11/194,789

(22) Filed: Aug. 1, 2005

(51) Int. Cl. A61K 8/81; A61K 8/73

(52) U.S. Cl. 424/64; 424/70.13; 424/70.15

(57) ABSTRACT

Disclosed are liquid lipgloss compositions comprising an oil, a wax and aluminum starch octenyl succinate, and methods of making and using them.

### BACKGROUND OF THE INVENTION

Lip gloss is typically used to enhance natural features by adding color and shine to the lip area. Generally, lip gloss and other lip compositions contain a particulate material such as pigment or particulate fillers in an oil and/or wax base. See, e.g., U.S. Publication no. 2004/0161395.

The cosmetic industry has endeavored to provide long lasting lip gloss that retains shine. U.S. Pat. No. 5,747,017 teaches compositions that containing a dimethicone. The compositions taught in U.S. Pat. No. 6,309,629 contain a high weight percentage of gel base consisting of a non-polar liquid ester or oil and a copolymer gellant.

### SUMMARY OF THE INVENTION

A first aspect of the present invention is directed to a liquid lip gloss composition comprising an oil, a wax and aluminum starch octenyl succinate.

A second aspect of the present invention is directed to a method of applying make-up, comprising applying to the lips a liquid lip gloss composition comprising an oil, a wax and aluminum starch octenyl succinate.

The inventive compositions have advantages over commercial lip gloss formulations. Not only do they have higher gloss or shine, the gloss is renewable simply upon mechanical compression of the lips. While not intending to be bound by theory, Applicants believe that the presence of the aluminum starch octenyl succinate effectively entraps relatively large amounts of oil, and releases it over time upon compression. There is less migration (and/or bleeding and feathering) e.g., spreading of the composition above and below the lip line, and into fine lines and wrinkles around the lips.

### DETAILED DESCRIPTION OF THE INVENTION

As used herein, the compositions of the present invention are liquids, which as used herein, refer to compositions that have a measurable viscosity at room temperature. The viscosity of the lip gloss provides for certain desired flow characteristics, e.g., to prevent its leakage from the container, retain the color and other ingredients of the system in uniform suspension or dispersion, to allow for easy spreadability on lips, and retention on the lips for a reasonable length of time. In general, lipglosses of the present invention have a viscosity in the range of about 10 Pa·s (pascal seconds) to about 100 Pa·s (as measured by a Rheomat RM 80 at a shear rate of 200 s⁻¹). The lip gloss compositions of the present inventionare in contrast to compositions in the form of a stick, such as lipstick, that do not have a measurable viscosity at room temperature. See, e.g., U.S. Pat. No. 6,344,187.

Aluminum starch octenyl succinate is commercially available from National Starch under the tradename DryFlo®. It is present in the compositions of the present invention in amounts generally ranging from about 0.05% to about 20%, and preferably about 1.0% to about 10%, based on the total weight of the composition.

As disclosed herein, the term “liquid fatty phase” refers to a non-aqueous medium that is liquid at room temperature (25° C.) and atmospheric pressure (760 mmHg, i.e., 105 Pa), comprising at least oil or oily liquid that in general, are mutually compatible. In addition to providing shine, oily (oil soluble) liquids are included in the lip gloss of the present to provide desirable feel, spreadability, and other desirable characteristics. The inventive compositions may contain any cosmetically or dermatologically acceptable oil, chosen in particular from carbon-based, hydrocarbon-based, fluoro and/or silicone oils, of mineral, animal, plant or synthetic origin, alone or as a mixture, provided that they form a homogeneous and stable mixture and provided that they are compatible with the intended use.

The term “hydrocarbon-based oil” means an oil mainly comprising carbon and hydrogen atoms and possibly at least one functional group chosen from hydroxyl, ester, ether and carboxylic functional groups. For example, the oils may have a viscosity ranging from 0.5 to 300,000 centipoise (cps), further for example, from 50 to 50,000 cps, and even further for example, from 100 to 100,000 cps. Examples of hydrocarbon-based oils include hydrocarbon-based oils of animal origin, such as perhydrosqualene; hydrocarbon-based plant oils such as liquid triglycerides of fatty acids of from 4 to 24 carbon atoms, for instance heptanoic or octanoic acid triglyceride, or alternatively sunflower oil, maize oil, soybean oil, marrow oil, grapeseed oil, sesame seed oil, hazelnut oil, apricot oil, macadamia oil, castor oil, avocado oil, caprylic/capric acid triglycerides, for instance those sold by the company Stearineries Dubois or those sold by the company Dynamit Nobel under the names Miglyol 810, 812 and 818, jojoba oil and shea butter; linear and branched hydrocarbons of mineral or synthetic origin, for example, liquid paraffin and derivatives thereof, petroleum jelly, polydecenes, polybutenes and hydrogenated polyisobutene, for example Parleam™; esters of lanolic acid, of oleic acid, of lauric acid or of stearic acid; fatty esters, such as isopropyl myristate, isopropyl palmitate, butyl stearate, hexyl laurate, diisopropyl adipate, isononyl isononate, 2-ethylhexyl palmitate, 2-hexyldecyl laurate, 2-octyldecyl palmitate, 2-octyldodecyl myristate or lactate, 2-octyldodecyl stearate, 2-octyldodecyl erucate, bis(2-ethylhexyl) succinate, diisostearyl malate, glyceryl triisostearate or diglyceryl triisostearate, isopropyl myristate, isostearyl isostearate and tridecyl trimellitate; higher fatty acids, such as myristic acid, palmitic acid, stearic acid, behenic acid, oleic acid, linoleic acid, linolenic acid or isostearic acid; higher fatty alcohols (e.g., having from 10-26 carbon atoms) such as ketanol, stearyl alcohol, oleyl alcohol, linoleyl alcohol, linolenyl alcohol, isostearyl alcohol or octyldodecanol; hydroxylated esters, for instance isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate.

The oils may include volatile and non-volatile oils. The volatility of the solvents/oils can be determined using the evaporation speed as set forth in U.S. Pat. No. 6,338,839. The inventive compositions may contain one or more volatile silicone oils. Examples of such volatile silicone oils include linear or cyclic silicone oils having a viscosity at room temperature less than or equal to 6 centistokes (cSt) and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils that may be used include KF 96A of 6 cSt viscosity, a dimethylpolysiloxane commercial product from Shin Etsu having a flash point of 94° C. Preferably, the volatile silicone oils have a flash point of at least 40° C. Non-limiting examples of volatile silicone oils are listed in Table 1 below.

TABLE 1

| | Flash Point | Viscosity |
|---|---|---|
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or D4) | | |
| Dodecamethylcyclohexasiloxane (D6) | 93 | 7 |
| Decamethyltetrasiloxane(L4) | 63 | 1.7 |
| KF-96 A from Shin Etsu | 94 | 6 |
| PDMS (polydimethylsiloxane) DC 200 | 56 | 1.5 |
| (1.5 cSt) from Dow Corning | | |
| PDMS DC 200 (2 cSt) from Dow Corning | 87 | 2 |
| PDMS DC 200 (5 cSt) from Dow Corning | 134 | 5 |
| PDMS DC 200 (3 St) from Dow Corning | 102 | 3 |

Further, a volatile linear silicone oil may be employed in the compositions of the present invention. Suitable volatile linear silicone oils include those described in U.S. Pat. No. 6,338,839 and WO03/042221. Another example is decamethyltetrasiloxane.

The composition may contain one or more non-silicone volatile oils such as volatile hydrocarbon oils, alcohols, volatile esters and volatile ethers. Examples of such volatile non-silicone oils include volatile hydrocarbon oils having from 8 to 16 carbon atoms and their mixtures, and in particular branched C₈ to C₁₆ alkanes such as C₈ to C₁₆ isoalkanes (also known as isoparaffins), isododecane, isodecane, isohexadecane, and for example, the oils sold under the trade names of Isopar or Permethyl, the C₈ to C₁₆ branched esters such as isohexyl or isodecyl neopentanoate, and their mixtures. Preferably, the volatile non-silicone oils have a flash point of at least 40° C.

Non-limiting examples of volatile non-silicone volatile oils are given in Table 2 below.

TABLE 2

| Compound | Flash Point (° C.) |
|---|---|
| Isododecane | 43 |
| Isohexadecane | 102 |
| Isodecyl Neopentanoate | 118 |
| Propylene glycol n-butyl ether | 60 |
| Ethyl 3-ethoxypropionate | 58 |
| Propylene glycol methylether acetate | 46 |
| Isopar L (isoparaffin C₁₁-C₁₃) | 62 |
| Isopar H (isoparaffin C₁₁-C₁₂) | 56 |

Examples of other silicone oils that may be used in the invention include non-volatile linear polydimethylsiloxanes (PDMSs) that are liquid at room temperature; polydimethylsiloxanes that may be substituted with fluoro groups, functional groups such as hydroxyl, thiol or amine groups, aliphatic (e.g., alkyl) groups or aromatic (e.g., phenyl) groups, which are pendent and/or at the end of a silicone chain, these groups each containing from 2 to 24 carbon atoms; phenylsilicones, for instance phenyl trimethicones, phenyl dimethicones, trimethyl pentaphenyl trisiloxane, tetramethyl tetraphenyl trisiloxane, phenyl trimethylsiloxydiphenylsiloxanes (e.g., DC555 from Dow Corning), diphenyl dimethicones, diphenyl methyldiphenyl trisiloxanes and 2-phenylethyl trimethylsiloxysilicates. Other examples of silicone oils include polysiloxanes modified with fatty acids, fatty alcohols or polyoxyalkylenes, fluorosilicates and perfluoro oils.

Low viscosity oils (generally from about 5 or 10 cps at 25° C., and up to about 100 cps, preferably up to about 50 cps, at 25° C.), high viscosity oils (at least about 100 cps, preferably at least about 150 cps (at 25° C.) and up to about 10,000, preferably up to about 1,000 cps (at 25° C.)), and mixtures thereof can be used. Low viscosity oils are preferred. Representative of low viscosity oil (viscosity in the range of 5 to 15 cps at 25° C. is isononyl isononanoate. Other suitable low viscosity oils include, for example, octyl palmitate, diioctylmaleate, octyldedecanol, PEG-4 diheptanoate, isononylnonoate, coco-dicaprylate/caprate, polyglyceryl-3-diisostearate, cetyl alcohol, isocetyl alcohol, oleyl alcohol, cetyl acetate, acetylated lanolin alcohol, and the like. Examples of high viscosity oils (viscosities in the range of 100 to 1,000 cps at 25° C.) include lanolin oil, sesame seed oil, glyceryl trioctanoate, tridecyl trimellitate, castor oil, caprylic/capric triglyceride, corn oil, mineral oil, hydrogenated polyisobutene, polybutene polyvinylpyrrolidone (PVP)/hexadecene, diisoarachidyl dilinoleate, diisopropyl malate (DISM) and trioctyldodecyl citrate.

Due to the presence of the aluminum starch octenyl succinate, compositions of the present invention may contain relatively more oil than other lipstick formulations. Thus, the amount of oil is above about 30% by weight, and generally ranges from about 50% to about 90% or more, and preferably from about 60% to about 80% by weight of the composition.

For the purposes of the present invention, a wax is a lipophilic fatty compound, which is solid at room temperature (25° C.) with a reversible solid/liquid change of state, having a melting point of greater than 45° C. and in some embodiments, greater than 55° C., which may be up to 200° C., and having an anisotropic crystal organization in the solid state.

For the purposes of the invention, the waxes are those generally used in cosmetics and dermatology. A variety of waxes may be useful, including waxes of animal origin, waxes of plant origin, waxes of mineral origin and waxes of synthetic origin. Examples of waxes of animal origin include beeswaxes, lanolin waxes and Chinese insect waxes. Examples of waxes of plant origin include rice waxes, carnauba wax, candellila wax and ouricurry wax, cork fibre waxes, sugar cane waxes, Japan waxes, sumach wax and cottonseed wax. Examples of waxes of mineral origin include paraffins, microcrystalline waxes (microwax), montan waxes and ozokerites. Examples of waxes of synthetic origin include polyolefin waxes, e.g., polyethylene waxes, synthetic beeswax, waxes obtained by Fischer-Tropsch synthesis, waxy copolymers and their esters, and silicone and fluoro waxes. Representative silicone waxes include alkyl, alkoxy or esters of poly(di)methylsiloxane silicones, which are polymers that comprise repeating dimethylsiloxy units in combination with alkyl siloxy units wherein the long chain alkyl is generally a fatty chain that provides a wax-like character to the silicone. Such silicones include, but are not limited to stearoxydimethicone, behenoxy dimethicone, stearyl dimethicone, cetearyl dimethicone, and the like which are solid at 40° C. In some instances, waxes of synthetic origin are preferably used for reasons of greater reproducibility than waxes of natural origin. Alternatively, hydrogenated oils of animal or plant origin may be used. Examples include hydrogenated jojoba waxes and hydrogenated oils which are obtained by catalytic hydrogenation of fats composed of a C₈-C₃₂ linear or nonlinear fatty chain, hydrogenated sunflower oil, hydrogenated castor oil, hydrogenated copra oil, hydrogenated lanolin and hydrogenated palm oils.

The amount of wax in the compositions generally ranges from about 0.5% to about 15%, preferably from about 1.0 to about 10% by weight based on the total weight of the composition.

In addition to wax, the compositions of the present invention may contain a structuring polymer. Examples of suitable structuring polymers are disclosed in U.S. Pat. Nos. 5,783,657 and 6,402,408. Specifically, the disclosed polymers are represented by the following formula (I): 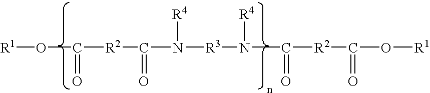
in which:
n is an integer which represents the number of amide units such that the number of ester groups present in the structuring polymer ranges from 10% to 50% of the total number of all the ester groups and all the amide groups comprised in the structuring polymer;
R¹, which are identical or different, are each chosen from alkyl groups comprising at least 4 carbon atoms and alkenyl groups comprising at least 4 carbon atoms; R², which are identical or different, are each chosen from C₄ to C₄₂ hydrocarbon-based groups with the proviso that at least 50% of R² are chosen from C₃₀ to C₄₂ hydrocarbon-based groups; R³, which are identical or different, are each chosen from organic groups comprising atoms chosen from carbon atoms, hydrogen atoms, oxygen atoms and nitrogen atoms with the proviso that R³ comprises at least 2 carbon atoms;
and R⁴, which are identical or different, are each chosen from hydrogen atoms, C₁ to C₁₀ alkyl groups and a direct bond to group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

In the present invention, n can be an integer ranging from 1 to 5. In the present invention, R¹, which are identical or different, can each be chosen from C₁₂ to C₂₂ alkyl groups, such as from C₁₆ to C₂₂ alkyl groups.

In the present invention, R², which are identical or different, can each be chosen from C₁₀ to C₄₂ alkyl groups. At least 50% of R², which are identical or different, can each be chosen from groups comprising from 30 to 42 carbon atoms. At least 75% of R², which are identical or different, can each be chosen from groups comprising from 30 to 42 carbon atoms. In the two aforementioned embodiments, the remaining R², which are identical or different, can each be chosen from C₄ to C₁₉ groups, such as C₄ to C₁₂ groups.

The substituent R³, which can be identical or different, can each be chosen from C₂ to C₃₆ hydrocarbon-based groups and polyoxyalkylene groups. In another example, R³, which can be identical or different, can each be chosen from C₂ to C₁₂ hydrocarbon-based groups. In another embodiment, R⁴, which can be identical or different, can each be chosen from hydrogen atoms. As used herein, hydrocarbon-based groups may be linear, cyclic or branched and saturated or unsaturated. The hydrocarbon-based groups can be aliphatic or aromatic.

Non-limiting examples of the structuring polymer that may be used in the compositions of the present include the products sold by the Arizona Chemical Co. under the names Uniclear 80 and Uniclear 100. These are sold, respectively, in the form of an 80% (in terms of active material) gel in a mineral oil and a 100% (in terms of active material) gel. These polymers have a softening point ranging from 88° C. to 94° C., and may be mixtures of copolymers derived from monomers of (i) C₃₆ diacids and (ii) ethylenediamine, and have a weight-average molecular mass of about 6000. Terminal ester groups result from esterification of the remaining acid end groups with at least one alcohol chosen from cetyl alcohol and stearyl alcohol. A mixture of cetyl and stearyl alcohols is sometimes called cetylstearyl alcohol. As disclosed in the '408 patent, these polymers are advantageously used in combination with at least one amphiphilic compound which is liquid at room temperature and which has an HLB value of less than 8.

Examples of non-wax structuring polymers are also disclosed in U.S. Patent Publication no. 20050008599. Further examples are the silicone-polyamide copolymers disclosed in U.S. Patent Publication no. 20040170586. Amounts of the non-wax structuring polymer generally range from about 0.1% to about 20% by weight of the composition.

The compositions of the present invention may also contain at least one fatty compound that is pasty at room temperature. For the purposes of the invention, the expression “pasty fatty substance” means a compound with a melting point ranging from 25 to 60° C., preferably from 30 to 45° C. and/or a hardness ranging from 0.001 to 0.5 MPa and preferably from 0.005 to 0.4 MPa.

The melting point values correspond to the melting point measured using a differential scanning calorimeter (DSC), such as the calorimeter sold under the name DSC 2920 by the company TA Instruments, with a temperature rise of 5 or 10° C. per minute. (The melting point considered is the point corresponding to the temperature of the most endothermic peak in the thermogram).

The hardness is measured according to a method of penetration of a probe into a sample of compound and in particular using a texture analyzer (for example the TA-XT2i from Rheo) equipped with a stainless steel cylinder 2 mm in diameter. The hardness measurement is performed at 20° C. at the center of 5 samples. The cylinder is introduced into each sample at a pre-speed of 1 mm/sec and then at a measuring speed of 0.1 mm/sec, the depth of penetration being 0.3 mm. The hardness value recorded is that of the maximum peak of the applied force.

Preferably, these fatty substances are hydrocarbon-based compounds, optionally of polymeric type; they may also be chosen from hydrocarbon-based compounds, silicone compounds and/or fluoro compounds, and mixtures thereof. Among the pasty compounds that may be mentioned are lanolins and lanolin derivatives, for instance acetylated lanolins or oxypropylenated lanolins, with a viscosity from 18 to 21 Pa·s and preferably 19 to 20.5 Pa·s, and/or a melting point from 30 to 45° C., and mixtures thereof. Esters of fatty acids or of fatty alcohols may also be used, especially those containing 20 to 65 carbon atoms (melting point of about 20 to 35° C. and/or viscosity at 40° C. ranging from 0.1 to 40 Pa·s), for instance triisostearyl or cetyl citrate; arachidyl propionate; polyvinyl laurate; cholesterol esters, for instance triglycerides of plant origin such as hydrogenated plant oils, viscous polyesters, for instance poly(12-hydroxystearic acid), and mixtures thereof. Triglycerides of plant origin that may be used include hydrogenated castor oil derivatives, such as “Thixin R” from Rheox.

Mention may also be made of silicone pasty fatty substances such as polydimethylsiloxanes (PDMS) containing pendant chains of the alkyl or alkoxy type containing from 8 to 24 carbon atoms, and having a melting point of 20-55° C., for instance stearyl dimethicones, especially those sold by the company Dow Corning under the trade names DC2503 and DC2-5514, and mixtures thereof. Another suitable pasty fatty substance is bis-digylcerylpolyacyladipate-2.

The pasty fatty substance may be present in an amount of from about 0.1 to about 70% by weight, and preferably from about 5 to about 50% by weight of the composition.

The inventive compositions may contain film-forming polymer, which is compatible with the oil/wax phase and which forms a film after application to the lips. Suitable polymers include homo- and copolymers of polyvinylpyrrolidone (PVP) and vinyl pyrrolidone (VP), e.g., vinyl pyrrolidone (VP)/hexadecene copolymer, PVP/hexadecene copolymer (an alkylated polyvinyl pyrrolidone copolymer), and VP/eicosene copolymer, trimethylsiloxysilicate (e.g., SR1000 from General Electric), resin MK (polymethylsilsesquioxane), silicone acrylates (e.g., KP 550 from Shin-Etsu) and acrylates copolymer. Other film-forming polymers that may be useful in the present invention are disclosed in co-owned patent application no. ______, filed of even date herewith (attorney docket no. (Loreal 3.0-095), the relevant contents of which are hereby incorporated by reference. The polymer may be present in the compositions in an amount generally ranging from 0 to about 50% by weight, and in some embodiments, about 10% to about 40% by weight, and in some other embodiments, from about 20% to about 35% by weight.

The inventive compositions, in preferred embodiments, will contain at least one colorant. Colorants are typically chosen from the lipophilic dyes, hydrophilic dyes, traditional pigments, and nacres usually used in cosmetic or dermatological compositions, and mixtures thereof. The colorant may have any shape, such as, for example, spheroidal, oval, platelet, irregular, and mixtures thereof. Pigments may optionally be surface-treated e.g., with silicones, perfluorinated compounds, lecithin, and amino acids.

The liposoluble dyes include, for example, Sudan Red, D&C Red 17, D&C Green 6, β-carotene, soybean oil, Sudan Brown, D&C Yellow 11, D&C Violet 2, D&C Orange 5 and quinoline yellow.

The pigments may be chosen from white pigments, colored pigments, inorganic pigments, organic pigments, coated pigments, uncoated pigments, pigments having a micron size and pigments not having a micron size. Among the inorganic pigments that may be mentioned are titanium dioxide, zirconium oxide, zinc oxide, cerium oxide, chromium oxide, manganese violet, ultramarine blue, chromium hydrate, and ferric blue. Among the organic pigments which may be mentioned are carbon black, pigments of D&C type, lakes based on cochineal carmine, lakes based on barium, lakes based on strontium, lakes based on calcium, and lakes based on aluminium.

The nacreous pigments may, for example, be chosen from white nacreous pigments such as mica coated with titanium and mica coated with bismuth oxychloride, colored nacreous pigments such as titanium mica with iron oxides, titanium mica with, for example, ferric blue and/or chromium oxide, titanium mica with an organic pigment of the type mentioned above, as well as nacreous pigments based on bismuth oxychloride, interferential pigments, and goniochromatic pigments.

The pigments can also be spherical scattering agents such as spherical powders that achieve a soft focus look. Examples include calcium aluminum borosilicate, PMMA, polyethylene, polystyrene, methyl methacrylate crosspolymer, nylon-12, ethylene/acrylic acid copolymer, boron nitride, Teflon, or silica.

Colorants can generally be present in an amount ranging from about 0.01% to about 30% relative to the total weight of the composition.

The compositions of the present invention may also contain dispersion enhancing agents such as the polysaccharide resin KM13®, available from KAMA International Corp. (Duluth, Ga.).

The compositions may further comprise at least one filler. As used herein, the term “filler” refers to any particle that is solid at room temperature and atmospheric pressure, which does not react chemically with the various ingredients of the composition and which is insoluble in these ingredients, even when these ingredients are raised to a temperature above room temperature and in particular to their softening point or their melting point. The filler may be absorbent, i.e., capable in particular of absorbing the oils of the composition and also the biological substances secreted by the skin. The filler may be surface-treated, e.g., with lecithin, silicones, amino acids, fatty acids, fatty alcohols, or metallic soaps e.g., to make it lipophilic, and/or may be porous so as to absorb the sweat and/or sebum secreted by the skin.

The filler may be chosen from inorganic and organic fillers, and may have any shape such as lamellar, spherical and/or oblong. Examples of fillers include talc, mica, fumed silica, kaolin, poly-β-alanine powders, acrylic polymer powders (such as acrylic acid copolymer powder sold by Dow Corning as Polytrap®), lauroyllysine, bismuth oxychloride, starch, starch derivatives, hollow polymer microspheres (such as those hollow polymer microspheres formed from polyvinylidene chloride and acrylonitrile, for instance the product sold by Nobel Industrie as Expancel®), and polymerized silicone microspheres (such as those polymerized silicone microspheres sold by Toshiba as Tospearl®), precipitated calcium carbonate, magnesium carbonate and hydrocarbonate, hydroxyapatite, ceramic microcapsules, polyester particles and coated elastomers such as products sold under the denomination KSP (KSP 100, KSP 200, KSP 300) sold by Shin Etsu and/or those described in U.S. Pat. No. 5,538,793.

In general, fillers and powders together (exclusive of aluminum starch octenyl succinate) constitute no more than about 30%, preferably about 20% and more preferably about 15% of the total weight of the composition.

The compositions of the present invention may comprise a conditioning agent selected from the group consisting of humectants, moisturizers, skin conditioners or emollients. A variety of these materials can be employed and each can be present in an amount generally ranging from about 0.01% to about 20% by weight of the composition. These materials include, but are not limited to, guanidine, urea, glycolic acid and glycolate salts (e.g. ammonium and quaternary alkyl ammonium), salicylic acid; lactic acid and lactate salts (e.g., ammonium and quaternary alkyl ammonium), aloe vera in any of its variety of forms (e.g., aloe vera gel), polyhydroxy alcohols such as sorbitol, glycerol, hexanetriol, butanetriol, propylene glycol, butylene glycol, hexylene glycol and the like, polyethylene glycols, sugars (e.g., melibiose) and starches, sugar and starch derivatives (e.g., alkoxylated glucose, fructose), hyaluronic acid, lactamide monoethanolamine and acetamide monoethanolamine.

Water may be present in the compositions of the present invention, generally in amounts up to 30% by weight. In these embodiments, the compositions are in the form of a water-in-oil (W/O) emulsion. Thus, they contain at least one surfactant. Surfactants typically employed in the compositions of the present invention include amphoteric, anionic, cationic and nonionic surfactants. See, e.g., Encyclopedia of Chemical Technology, KIRK-OTHMER, volume 22, pp. 333-432, 3rd edition, 1979, Wiley, for the definition of the properties and (emulsifying) functions of the surfactants, in particular pp. 347-377 of this publication regarding anionic and nonionic surfactants. Examples of surfactants useful in the compositions of the invention include as nonionic surfactants, fatty acids, fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C₁-C₆ alkyl glucose fatty esters, and as anionic surfactants, C₁₆-C₃₀ fatty acids neutralized by amines, ammonia or the alkali metal salts thereof. Examples of amphoteric surfactants include betaines, sultaines, hydroxysultaines, alkyl amphodiacetates, alkyl amphodipropionates, and imidazolines, or salts thereof. Other fatty acid condensates such as those formed with amino acids, proteins, and the like are suitable as well. Specific examples include cocamphodipropionate, e.g., Miranol C2M-SF (disodium cocamphodipropionate), in its salt-free form, available from Rhone-Poulenc, and Crosultaine C-50 (cocamidopropyl hydroxysultaine), available from Croda. Examples of cationic surfactants include quaternary amines, amine oxides and amines, e.g., alkyl amines, alkyl imidazolines, ethoxylated amines, quaternary compounds, and quaternized esters.

In preferred embodiments, the compositions of the present invention are substantially anhydrous (wherein water constitutes no more than about 2% by weight of the composition) or anhydrous (in which case, there is no added water).

The composition can also contain other cosmetic or determatologically acceptable adjuvants, which are optional components, including, but not limited to, organic oil soluble sunscreens, such as octyl methoxycinnamate, octocrylene and avobenzone; inorganic, e.g., particulate sunscreens such as titanium dioxide and zinc oxide; oil-soluble antioxidants and/or preservatives such as BHT; chelating agents such as disodium EDTA; fragrances (such as pinene); algae, flavoring agents; waterproofing agents (such as PVP/eicosene copolymer); and oil-soluble actives, such as vitamins e.g., tocopherol and its derivatives or retinol and its derivatives; and the like. In embodiments that contain water, water-soluble adjuvants may be included.

The lip gloss compositions can be prepared and packaged (e.g., with a brush-type applicator) in accordance with standard techniques.

The following example further illustrates the present invention. It is not intended to be limiting in any way. Unless otherwise indicated, all parts are by weight.

### EXAMPLE 1

### Lip Gloss Composition

| Phase | | Chemical Names | % |
|---|---|---|---|
| A | Oil | Diisopropyl Dimer Dilinoleate | 2.87 |
| | Phase | VP/Hexadecene Copolymer | 30 |
| | | Polybutene | 22 |
| | | Bis-Diglyceryl Polyacyladipate-2 | 15 |
| | | Stearyl Heptanoate | 5 |
| | | Isoeicosane | 3.25 |
| | | Oil Phase | 78.12 |
| B | Waxes | Synthetic Beeswax | 6.00 |
| | | Waxes | 6.00 |
| C | Colorants | | 12.78 |
| | and | | |
| | Fillers | | |
| D | | Aluminum Starch Octenylsuccinate | 3 |
| | | | 5.25 |
| | | | 4 |
| E | Fragrance | | 0.1 |
| | | | 100.00 |

To prepare the lipgloss, wax was melted at 85° C. In a separate beaker, colorant was added to phase A ingredients with mixing and grinding to achieve a homogeneous dispersion. The dispersion was then added to the melted wax, while maintaining temperature of 85° C., followed by addition of phase D. Mixing was slowed, and cooling was initiated. When the temperature reached 40° C., phase E was added with mixing, and the composition was poured into the appropriate package.

All publications cited in the specification are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A liquid lip gloss composition, comprising aluminum starch octenyl succinate, an oil and a wax.

2. The composition of claim 1, wherein said aluminum starch octenyl succinate is present in an amount of about 0.05% to about 20% by weight of the composition.

3. The composition of claim 1, wherein said aluminum starch octenyl succinate is present in an amount of about 1.0% to about 10% by weight of the composition.

4. The composition of claim 1, wherein said oil is present in an amount of about 50% to about 90% by weight of the composition.

5. The composition of claim 1, wherein said oil is present in an amount of about 60% to about 80% by weight of the composition.

6. The composition of claim 1, wherein said oil comprises a low-viscosity oil.

7. The composition of claim 1, wherein said oil comprises a volatile oil.

8. The composition of claim 1, wherein said wax is present in an amount of about 0.5 to about 15% by weight of the composition.

9. The composition of claim 1, further comprising an oil-soluble film-forming polymer.

10. The composition of claim 9, wherein said polymer is present in an amount up to about 50% by weight of the composition.

11. The composition of claim 9, wherein the film forming polymer comprises PVP/hexadecene.

12. The composition of claim 1, further comprising water.

13. The composition of claim 12, further comprising a surfactant.
